(19) United States
(12) Patent Application Publication
(10) Pub. No.: US 2023/0001160 A1
(43) Pub. Date: Jan. 5, 2023

(54) Instrument Advancement Device Configured for Septum Engagement

(71) Applicant: Becton, Dickinson and Company, Franklin Lakes, NJ (US)

(72) Inventors: Erica E. Neumann, Salt Lake City, UT (US); Megan Scherich, Salt Lake City, UT (US); Jonathan Karl Burkholz, Salt Lake City, UT (US); Curtis H. Blanchard, Riverton, UT (US); John Lackey, West Valley City, UT (US); Weston F. Harding, Lehi, UT (US); Lisa Bailey, Salt Lake City, UT (US); Ralph L. Sonderegger, Farmington, UT (US)

(21) Appl. No.: 17/851,641

(22) Filed: Jun. 28, 2022

Related U.S. Application Data
(60) Provisional application No. 63/218,107, filed on Jul. 2, 2021.

(51) Int. Cl. A61M 25/09

(52) U.S. Cl. CPC A61M 25/09041

(57) ABSTRACT

An instrument advancement device may include a housing, which may include a slot. The instrument advancement device may be configured to couple to a catheter assembly. The instrument advancement device may include an advancement element, which may extend through the slot and may be configured to move linearly along the slot between a retracted position and an advanced position. In response to movement of the advancement element from a retracted position to an advanced position, an instrument of the instrument advancement device may be advanced beyond a distal end of the housing and may be configured to open a path through an occlusion in the catheter assembly or vasculature. The instrument advancement device may include a septum, which may be engaged to facilitate sealing of the housing and/or prevent buckling of the instrument.

### CROSS-REFERENCE TO RELATED APPLICATION

The present application claims priority to U.S. Provisional Application Ser. No. 63/218,107, entitled “Instrument Advancement Device Configured for Septum Engagement”, filed Jul. 2, 2021, the entire disclosure of which is hereby incorporated by reference in its entirety.

### BACKGROUND OF THE INVENTION

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient.

A common type of catheter device includes a catheter that is over-the-needle. As its name implies, the catheter that is over-the-needle may be mounted over an introducer needle having a sharp distal tip. A catheter assembly may include a catheter adapter, the catheter extending distally from the catheter adapter, and the introducer needle extending through the catheter. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient.

In order to verify proper placement of the introducer needle and/or the catheter in the blood vessel, a clinician generally confirms that there is “flashback” of blood in a flashback chamber of the catheter assembly. Once placement of the needle has been confirmed, the clinician may temporarily occlude flow in the vasculature and remove the needle, leaving the catheter in place for future blood withdrawal or fluid infusion.

Infusion and blood withdrawal using the catheter may be difficult for several reasons, particularly when an indwelling time of the catheter increases. A fibrin sheath or thrombus may form on an internal surface of the catheter assembly, an external surface of the catheter assembly, or within the vasculature near the distal tip of the catheter. The fibrin sheath or thrombus may block or narrow a fluid pathway through the catheter, which may impair infusion and/or collection of a high-quality blood sample.

The subject matter claimed herein is not limited to embodiments that solve any disadvantages or that operate only in environments such as those described above. Rather, this background is only provided to illustrate one example technology area where some implementations described herein may be practiced.

### SUMMARY OF THE INVENTION

The present disclosure relates generally to vascular access devices and related systems and methods. In particular, in some embodiments, the present disclosure relates to an instrument advancement device that engages a septum to facilitate sealing and separation from a fluid pathway. In some embodiments, the fluid pathway may be in fluid communication with a proximal portion of the housing until the advancement element is moved to an advanced position, which may result in the septum sealing off the fluid pathway from the proximal portion of the housing. In some embodiments, the instrument advancement device may also reduce buckling of an instrument of the instrument advancement device.

In some embodiments, the instrument advancement device may be configured to advance the instrument into a catheter assembly and/or through the catheter assembly into vasculature of a patient. In some embodiments, the instrument may be advanced through a catheter of the catheter assembly to push past any occlusions in the catheter or the vasculature (e.g., thrombus or fibrin sheath at a tip of the catheter, vein collapse, or valves) to create a clear pathway for fluid flow. In some embodiments, the instrument may reduce or remove occlusions, improving patency of the catheter for medication and fluid delivery, as well as blood acquisition, during a dwell time of the catheter. In some embodiments, the catheter that may be inserted into vasculature of the patient prior to advancing the instrument in the catheter assembly and may remain in the vasculature when the instrument is advanced via the instrument advancement device. In some embodiments, the catheter may include a peripherally-inserted central catheter, a midline catheter, or a peripheral intravenous catheter.

In some embodiments, the instrument advancement device may include a housing, which may include a proximal end, a distal end, and a slot disposed between the proximal end of the housing and the distal end of the housing. In some embodiments, the housing may include a lumen disposed between the proximal end of the housing and a distal end of the housing. In some embodiments, the housing may include a septum pocket disposed within the housing and configured to receive a septum.

In some embodiments, the instrument advancement device may include an advancement element extending through the slot and configured to move linearly along the slot between a retracted position and an advanced position. In some embodiments, the retracted position may correspond to an initial position and/or a position to which the advancement element may return after moving to the advanced position. In some embodiments, the instrument advancement device may include an instrument comprising a first end and a second end. In some embodiments, when the advancement element is moved linearly along the slot from the retracted position to the advanced position, the second end of the instrument may be advanced beyond the distal end of the housing.

In some embodiments, the instrument advancement device may include the septum, which may be coupled to the instrument. In some embodiments, when the advancement element is moved linearly along the slot from the retracted position and the advanced position, the septum may be configured to insert into the septum pocket to seal a portion of the lumen proximal to the septum.

In some embodiments, the distal end of the housing may include a connector configured to couple to the catheter assembly. In some embodiments, the instrument may include a guidewire. In some embodiments, the instrument delivery device may include an extension tube, which may extend from the distal end of the housing and may be distal to the septum pocket. In some embodiments, the septum may include an annular taper. In some embodiments, the septum pocket may include another annular taper. In some embodiments, a cross-section of the septum is an oval or circular shape. In some embodiments, a cross-section of the septum is a rectangular or square shape.

In some embodiments, the septum pocket may be disposed within the housing and configured to receive a split septum. In some embodiments, the split septum may be proximal to the septum pocket and spaced apart from the septum pocket. In some embodiments, in response to movement of the advancement element linearly along the slot, the split septum may be configured to insert into the septum pocket and the two sides of the split septum may be configured to press together to seal a portion of the lumen proximal to the septum.

In some embodiments, the instrument advancement device may include a spring, which may be disposed within the housing and/or proximate a proximal end of the split septum. In some embodiments, when the advancement element is moved linearly along the slot from the retracted position, the spring may be configured to compress the septum before the septum is inserted into the septum pocket.

In some embodiments, the septum may be secured within the housing, and a proximal end of the septum may include a tapered pocket. In some embodiments, the instrument advancement device may include a funnel-shaped element, which may include a distal column coupled to a proximal cone-shape. In some embodiments, the instrument may extend through the funnel-shaped element. In some embodiments, when the advancement element is moved linearly along the slot from the retracted position to the advanced position, the distal column may be configured to extend through the septum and the proximal cone-shape is disposed within the tapered pocket of the septum.

In some embodiments, the housing may include a support wall, which may include an opening configured to receive a guidewire therethrough. In some embodiments, the distal face of the septum may be in contact with the support wall. In some embodiments, the guidewire may include a first end and a second end. In some embodiments, when the advancement element is moved linearly along the slot from the retracted position to the advanced position, the second end of the guidewire may be advanced beyond the distal end of the housing.

In some embodiments, an outer diameter of the guidewire may increase in a proximal direction such that the guidewire compresses the septum a greater amount when the advancement element is in the retracted position than when the advancement element is in the advanced position. In some embodiments, a proximal face of the septum may include a lubricant. In some embodiments, when the advancement element is in the retracted position, the guidewire may extend through the septum and the outer diameter of the guidewire may be spaced apart from the septum.

In some embodiments, the instrument advancement device may include a first advancement element extending through the slot and configured to move linearly along the slot between a first retracted position and a first advanced position. In some embodiments, the instrument advancement device may include a second advancement element extending through the slot and configured to move linearly along the slot between a second retracted position and a second advanced position. In some embodiments, the second advancement element may be proximal to the first advancement element.

In some embodiments, a tube may be coupled to the first advancement element. In some embodiments, when the first advancement element is in the first advanced position, the tube may extend through the septum. In some embodiments, the guidewire may be coupled to or extend through the second advancement element and the tube. In some embodiments, the guidewire may include a first end and a second end, and when the second advancement element is moved linearly along the slot from the retracted position to the advanced position, the second end of the guidewire may be advanced beyond the distal end of the housing. In some embodiments, in response to the first advancement element being in the first retracted position, a distal end of the tube may be proximal to the septum. In some embodiments, the second advancement element may be disposed in the retracted position.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE DRAWINGS

Example embodiments will be described and explained with additional specificity and detail through the use of the accompanying drawings in which:

FIG. 1A is an upper perspective view of an example instrument advancement device, illustrating an example advancement element in an example retracted position, according to some embodiments;

FIG. 1B is a cross-sectional view of the instrument advancement device;

FIG. 1C is a cross-sectional view of the instrument advancement device along the line 1C-1C of FIG. 1A, according to some embodiments;

FIG. 1D is an enlarged view of a portion of FIG. 1C, according to some embodiments;

FIG. 1E is a cross-sectional view of the instrument advancement device along the line 1E-1E of FIG. 1A, according to some embodiments;

FIG. 2A is a cross-sectional view of the instrument advancement device, illustrating an example septum coupled to an example instrument, according to some embodiments;

FIG. 2B is a cross-sectional view of the instrument advancement device, illustrating the septum inserted into an example septum pocket, according to some embodiments;

FIG. 3A is a cross-sectional view of the instrument advancement device, illustrating an example split septum, according to some embodiments;

FIG. 3B is a cross-sectional view of the instrument advancement device, illustrating the split septum inserted into the septum pocket, according to some embodiments;

FIG. 4A is a cross-sectional view of the instrument advancement device, illustrating a funnel-shaped element, according to some embodiments;

FIG. 4B is a cross-sectional view of the instrument advancement device, illustrating the funnel-shaped element into an example tapered pocket, according to some embodiments;

FIG. 5A is a cross-sectional view of the instrument advancement device, illustrating an example support wall, according to some embodiments;

FIG. 5B is an enlarged cross-sectional view of a portion of FIG. 5A, illustrating the support wall, according to some embodiments;

FIG. 6A is a schematic diagram of the instrument when the advancement element is in the retracted position, illustrating a tapered outer diameter of the instrument, according to some embodiments;

FIG. 6B is a schematic diagram of the instrument when the advancement element is advanced distal to the retracted position or to an advanced position, according to some embodiments;

FIG. 6C is an enlarged view of a portion of FIG. 6A, according to some embodiments;

FIG. 6D is an enlarged view of a portion of FIG. 6B, according to some embodiments;

FIG. 6E is an enlarged view illustrating example multiple tapers, according to some embodiments;

FIG. 7A is a cross-sectional view of the instrument advancement device, illustrating an example first advancement element in a first advanced position and an example second advancement element in a second retracted position, according to some embodiments;

FIG. 7B is a cross-sectional view of the instrument advancement device, illustrating the first advancement element in the first advanced position and the second advancement element in a second advanced position, according to some embodiments; and

FIG. 7C is a cross-sectional view of the instrument advancement device, illustrating the first advancement element in a first retracted position and the second advancement element in the second retracted position, according to some embodiments.

### DETAILED DESCRIPTION

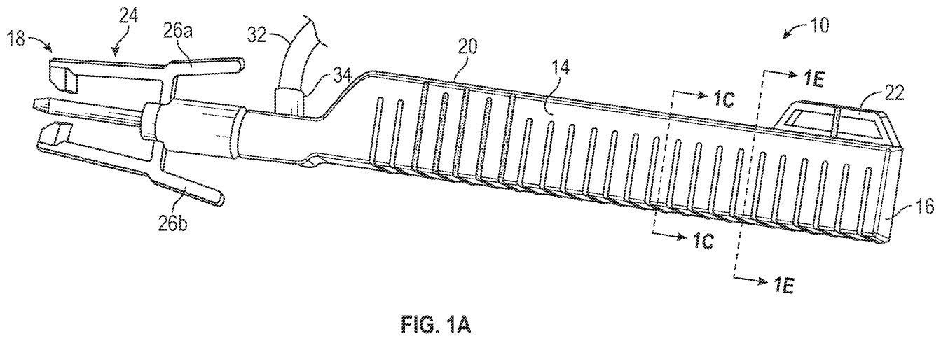

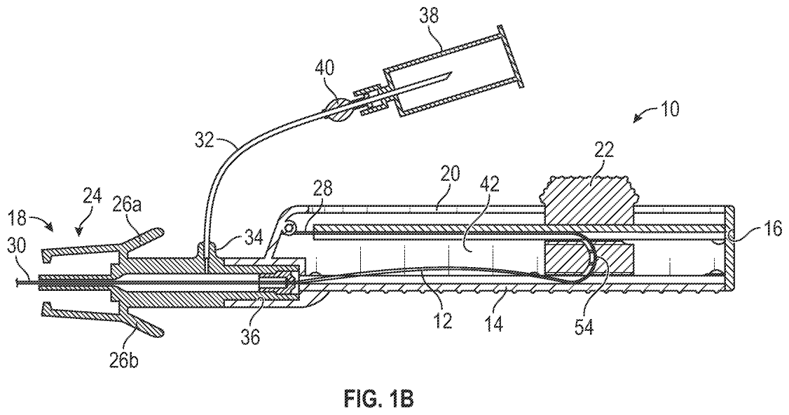

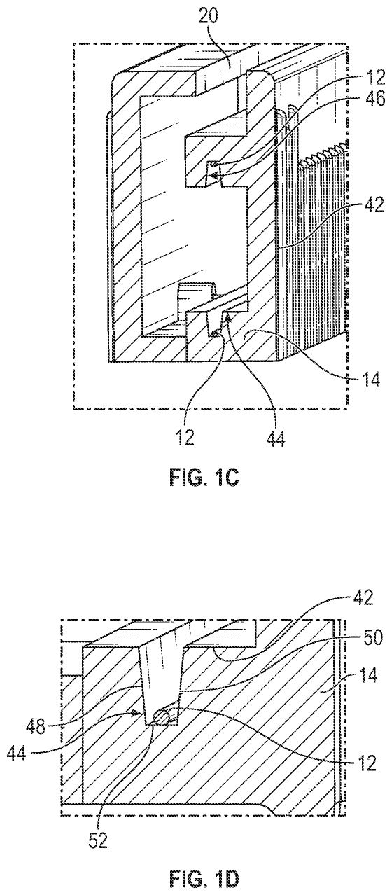

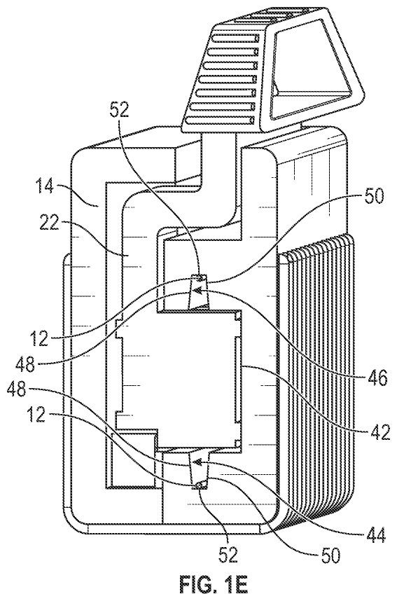

Referring now to FIGS. 1A-1E, in some embodiments, an instrument advancement device 10 may be configured to deliver an instrument 12 into and/or through a catheter of a catheter assembly. In some embodiments, the instrument 12 may be advanced through the catheter to push past any occlusions in the catheter or vasculature (e.g., thrombus or fibrin sheath at a tip of the catheter, vein collapse, valves, etc.) to create a clear pathway for fluid flow. In some embodiments, the instrument 12 may reduce or remove occlusions, improving patency of the catheter for medication and fluid delivery, as well as blood acquisition, during a dwell time of the catheter.

In some embodiments, the instrument 12 may include a guidewire, a probe, a guidewire or a probe with one or more sensors, or another suitable instrument. In some embodiments, the guidewire may be constructed of metal or another suitable material. In some embodiments, the sensors may be used for patient or device monitoring and may include sensors measuring pressure, temperature, pH, blood chemistry, oxygen saturation, flow rate, or another physiological property. In some embodiments, the catheter may include a peripheral intravenous (IV) catheter, a peripherally-inserted central catheter, or a midline catheter. In some embodiments, the catheter through which the instrument 12 may be delivered may have been previously inserted into vasculature of a patient and may be dwelling within the vasculature when the instrument 12 is advanced through the catheter.

In some embodiments, the instrument 12 may be disposed within a housing 14, which may be configured to protect the instrument 12 from damage and/or contamination from a surrounding external environment. In some embodiments, the housing 14 may be rigid or semi-rigid. In some embodiments, the housing 14 may be made of one or more of stainless steel, aluminum, polycarbonate, metal, ceramic, plastic, and another suitable material. In some embodiments, the housing 14 may include a proximal end 16, a distal end 18, and a slot 20. In some embodiments, the slot 20 may extend parallel to a longitudinal axis of the housing 14.

In some embodiments, the instrument advancement device 10 may include an advancement element 22, which may extend through the slot 20 and may be configured to move linearly along the slot 20 between a retracted position illustrated, for example, in FIG. 1A, and an advanced position. In some embodiments, the clinician may pinch or grasp the advancement element 22 to move the advancement element 22 between the retracted position and the advanced position.

In some embodiments, the distal end 18 of the housing 14 may include a connector 24. In some embodiments, the connector 24 may include opposing lever arms 26a, 26b. In some embodiments, distal ends of the opposing lever arms 26a, 26b may be configured to move apart from each other in response to pressure applied to proximal ends of the opposing lever arms 26a, 26b. In some embodiments, in response to removal of the pressure applied to the proximal ends of the opposing lever arms 26a, 26b, the distal ends may move closer to each other and clasp a portion of the catheter assembly, such as a needleless connector, another connector, or a proximal end of a catheter adapter, for example. In some embodiments, the connector 24 may include a blunt cannula or male luer configured to insert into the portion of the catheter assembly.

In some embodiments, the connector 24 may include any suitable connector. For example, the connector 24 may include a threaded male luer, a slip male luer, a threaded male luer with a spin lock, a threaded male luer with a removable blunt cannula snap connection, a slip male luer with a removable blunt cannula snap connection, or another suitable connector. In some embodiments, the connector 24 may include one or more bond pockets, which may each be configured to receive an extension tube. In some embodiments, the connector 24 may be monolithically formed as a single unit with a body of the housing 14 that includes the slot 20.

In some embodiments, the instrument 12 may include a first end 28 and a second end 30. In some embodiments, movement of the advancement element 22 from the retracted position to the advanced position may cause the second end 30 of the instrument 12 to be advanced beyond the distal end 18 of the housing 14. In some embodiments, moving the advancement element 22 to the advanced position may introduce the instrument 12 into the catheter assembly and/or through the catheter. In some embodiments, in response to the instrument 12 being introduced into the catheter assembly and/or through the catheter, the instrument 12 may access a fluid pathway of the catheter assembly and/or the vasculature of a patient.

In some embodiments, the catheter of the catheter assembly with significant dwelling time within the vasculature may be susceptible to narrowing, collapse, kinking, blockage by debris (e.g., fibrin or platelet clots), and adhering of a tip of the catheter to the vasculature. Thus, blood withdrawal using the catheter may be difficult. In some embodiments, the instrument 12 may have a diameter less than a diameter of the catheter of the catheter assembly to provide access to the vasculature of the patient without any additional needle sticks. In some embodiments, the instrument 12 may clear the pathway for collecting a blood sample. Thus, in some embodiments, the instrument advancement device 10 may be used for needle-free blood collection and/or fluid infusion.

In some embodiments, an extension tube 32 may be coupled to the instrument advancement device 10, and the extension tube 32 may be used for blood collection and/or fluid infusion. In some embodiments, the extension tube 32 may extend from a port 34 of the housing 14 or another portion of the housing 14.

In some embodiments, a septum 36 may be within the housing 14 to enable the instrument 12 to advance and/or retract while maintaining a closed fluid path. In some embodiments, the instrument 12 may be configured to extend through the septum 36. In some embodiments, the septum 36 may be disposed proximal to the port 34 and/or distal to the advancement element 22 in the advanced position. In some embodiments, the septum 36 may include silicone, rubber, an elastomer, or another suitable material. In some embodiments, the septum 36 may include a slit to accommodate the instrument 12 therethrough.

In some embodiments, a proximal end of the extension tube 32 may be coupled to a blood collection device 38. For example, the proximal end of the extension tube 32 may be integrated with a connector 40, which may be coupled to the blood collection device 38. In some embodiments, a needleless connector may be disposed between the connector 40 and the blood collection device 38. In some embodiments, the connector 40 and/or the port 34 may be coupled to an IV line or another fluidic connection.

In some embodiments, an inner surface 42 of the housing 14 may include one or more grooves. For example, the inner surface 42 may include a first groove 44 and/or a second groove 46. In some embodiments, the first groove 44 and/or the second groove 46 may be disposed within the housing 14 between the proximal end 16 and the distal end 18. In some embodiments, the instrument 12 may be disposed within the first groove 44 and/or the second groove 46. In some embodiments, the first groove 44 and/or the second groove 46 may include a side wall 48, another side wall 50 opposite the side wall, and a bottom 52 extending between the side wall 48 and the other side wall 50. In some embodiments, the first groove 44 and/or the second groove 46 may be open opposite the bottom 52. In some embodiments, the first groove 44 and/or the second groove 46 may be linear and/or configured to guide the instrument 12 as the instrument 12 is advanced distally and/or retracted proximally.

In some embodiments, the advancement element 22 may include an arc-shaped channel 54, which may be U-shaped. In some embodiments, the instrument 12 may extend and move through the arc-shaped channel 54. In some embodiments, the first end 28 of the instrument 12 may be fixed. In some embodiments, the first end 28 of the instrument may be fixed within the housing 14. In some embodiments, in response to movement of the advancement element 22 a first distance, the second end of the instrument 12 may be configured to advance distally a second distance. In some embodiments, the second distance may be twice the first distance. In some embodiments, the second distance may be more than twice the first distance. In these and other embodiments, the instrument 12 may extend through multiple U-shapes or other arc-shapes.

In some embodiments, the instrument advancement device 10 may include any suitable instrument advancement device that includes an advancement element configured to move linearly along a slot of a housing to advance and/or retract the instrument 12. For example, the first end 28 of the instrument 12 may be fixed to the advancement element 22, and in response to the advancement element 22 moving a distance, the second end 30 of the instrument 12 may move a distance equal to the distance such that advancement ratio between the instrument 12 and the advancement element 22 is 1:1. In some embodiments, because the first groove 44 and/or the second groove 46 are open opposite the bottom 52, the instrument 12 may tend to buckle in response to the advancement element 22 being advanced distally, as illustrated, for example, in FIG. 1B.

In some embodiments, the instrument advancement device 10 of FIGS. 1-7 may be similar or identical to the instrument advancement device 10 of U.S. patent application Ser. No. 17/709,980, filed Mar. 31, 2022, the entire disclosure of which is hereby incorporated by reference, in terms of one or more features and/or operation. For example, the advancement element 22 may be similar or identical to the housing 10 in U.S. patent application Ser. No. 17/709,980, in terms of one or more features and/or operation.

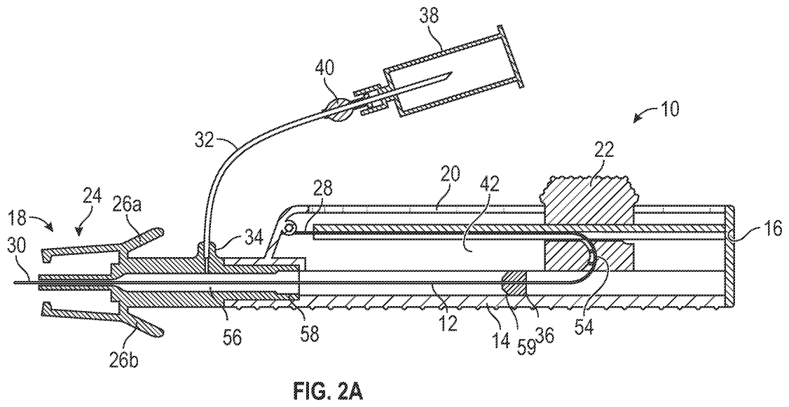

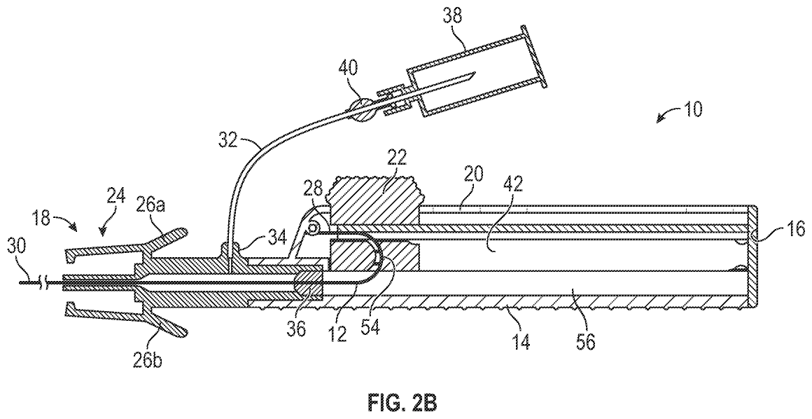

Referring now to FIGS. 2A-2B, in some embodiments, the housing 14 may include a lumen 56 disposed between the proximal end 16 of the housing 14 and the distal end 18 of the housing 14. In some embodiments, the housing 14 may include a septum pocket 58 disposed within the housing 14 and proximate the lumen 56. In some embodiments, the septum pocket 58 may be disposed in a wall of the housing 14. In some embodiments, the septum pocket 58 may be disposed within a proximal end of the connector 24 of the housing 14.

In some embodiments, the septum pocket 58 may be configured to receive the septum 36, which may be coupled to the instrument 12. In some embodiments, the septum 36 may be fixedly coupled or attached to the instrument 12 such that the septum 36 moves with the instrument 12. In some embodiments, the septum 36 may be fixedly coupled or attached to the instrument 12 via glue, an interference fit, etc.

In some embodiments, the weight of the septum 36 and/or positioning of the septum 36 within the first groove 44 may reduce buckling of the instrument 12 when the instrument 12 is advanced distally. In some embodiments, the septum 36 may be disposed within the first groove 44 and proximal to the septum pocket 58 when the advancement element 22 is in a retracted position. In some embodiments, the septum pocket 58 may be aligned with the first groove 44, which may facilitate the septum 36 entering the septum pocket 58 when the advancement element 22 is moved to the advanced position. In some embodiments, the septum pocket 58 may be proximal to a junction of the extension tube 32 with the connector 24 and/or the housing 14. In some embodiments, an absorbent material may be disposed in the housing 14 distal to the septum pocket 58 such that fluid that reaches the housing 14 before the septum 36 is engaged with the septum pocket 58 is absorbed and does not leak into the housing 14.

In some embodiments, in response to the instrument advancement device 10 being in the retracted position, an entirety of the instrument 12 may be disposed within the housing 14 and/or the advancement element 22 may be disposed at a proximal end of the slot 20. FIG. 2A illustrates the advancement element 22 moving between the retracted position and the advanced position, according to some embodiments. In some embodiments, when the advancement element 22 is in the advanced position, the advancement element 22 may be disposed at a distal end of the slot 20 and the instrument 12 may be fully advanced in a distal direction. In some embodiments, when the advancement element 22 is moved linearly along the slot 20 from the retracted position to the advanced position, the second end 30 of the instrument 12 may be advanced beyond the distal end 18 of the housing 14.

In some embodiments, when the advancement element 22 is moved linearly along the slot 20 from the retracted position and the advanced position, the septum 36 may be configured to insert into the septum pocket 58 to seal a portion of the lumen 56 proximal to the septum 36. In some embodiments, with the advancement element 22 in the advanced position and the instrument 12 advanced into and/or through the catheter assembly, blood may flow into the connector 24 and through the extension tube 32 for collection without entering a portion of the housing 14 proximal to the septum 36. In these and other embodiments, the instrument 12 may include a guidewire and thus may clear any obstructions that may interfere with blood collection.

In some embodiments, a shape of the septum pocket 58 may correspond to a shape of the septum 36 to facilitate sealing between the septum pocket 58 and the septum 36. For example, the septum 36 may include an annular taper 59. In some embodiments, the septum pocket 58 may include another annular taper. In some embodiments, a cross-section of the septum 36 may be an oval or circular shape which may be slightly larger than a cross-section of the lumen 56 at the septum pocket 58 so as to be in a press-fit, fill the cross-section of the lumen 56, and provide a seal. In some embodiments, a cross-section of the septum 36 may be a rectangular or square shape.

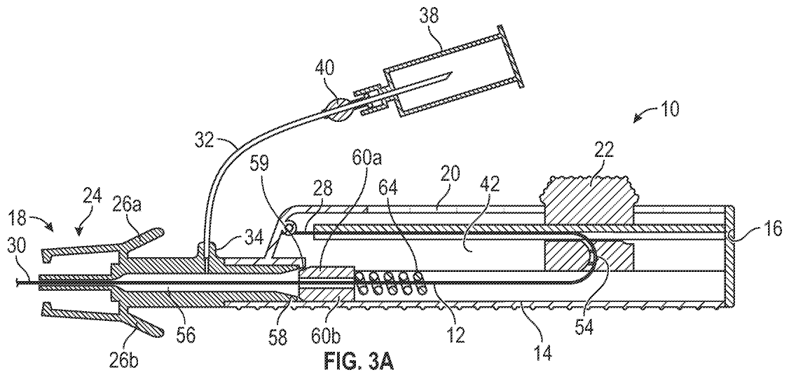

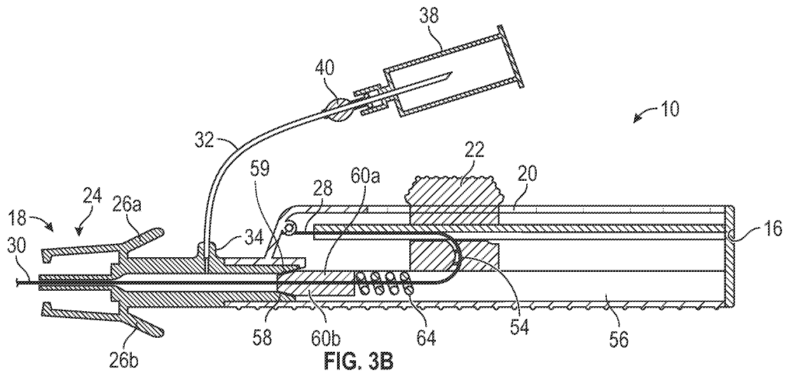

Referring now to FIGS. 3A-3B, in some embodiments, the septum pocket 58 may be disposed within the housing 14 and configured to receive a split septum 60, which may include two sides 60a, 60b. In some embodiments, the split septum 60 may include any suitable annular septum that includes an opening therethrough that has a diameter greater than an outer diameter of the instrument 12 such that friction between the split septum 60 and the instrument 12 is reduced or eliminated. In some embodiments, the opening may be formed by an inner surface of the split septum 60, which may be generally cylindrical or another suitable shape. In some embodiments, the split septum 60 may be proximal to the septum pocket 58 and spaced apart from the septum pocket 58. In some embodiments, in response to movement of the advancement element 22 linearly along the slot 20, the split septum 60 may be configured to insert into the septum pocket 58 and the two sides 60a, 60b of the split septum 60 may be configured to press together to seal a portion of the lumen 56 proximal to the split septum 60. In further detail, in some embodiments, in response to insertion of the split septum 60 into the septum pocket 58, the split septum 60 may be compressed such that the diameter of the opening extending through the split septum 60 may decrease and an annular seal is created around the instrument 12 by the inner surface of the split septum 60. In some embodiments, a distal end of the split septum 60 may include the annular taper 59.

In some embodiments, in response to movement of the advancement element 22 linearly along the slot 20 from the retracted position to the advanced position, the two sides 60a, 60b of the split septum 60 may be configured to contact the septum pocket 58 and press together to seal the portion of the lumen 56 proximal to the septum 36. In some embodiments, when the advancement element 22 is in the retracted position, the instrument 12 may be spaced apart from an inner surface of the split septum 60 forming the opening of the split septum 60, which may decrease friction on the instrument 12 and reduce buckling when the instrument 12 is advanced distally.

In some embodiments, when the advancement element 22 is in the advanced position, the advancement element 22 may be disposed at the distal end of the slot 20 or may be spaced part from the distal end of the slot 20 (as illustrated, for example, in FIG. 3B). In some embodiments, the instrument advancement device 10 may include a spring 64, which may be disposed within the housing 14 and/or proximate a proximal end of the split septum 60. In some embodiments, when the advancement element 22 is moved linearly along the slot 20 from the retracted position, the spring 64 may be configured to compress the split septum 60 before the split septum 60 is inserted into the septum pocket 58.

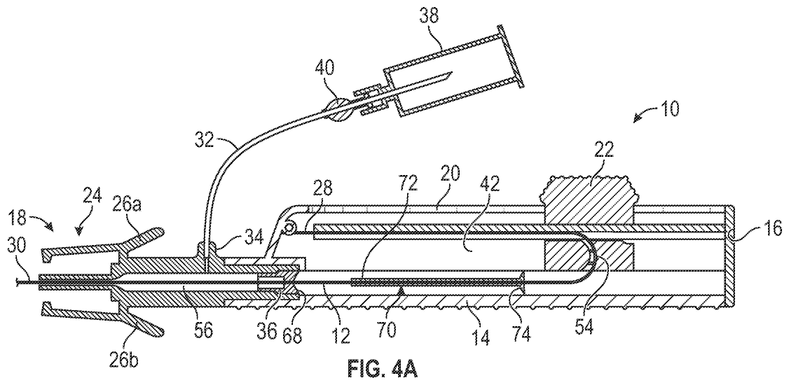

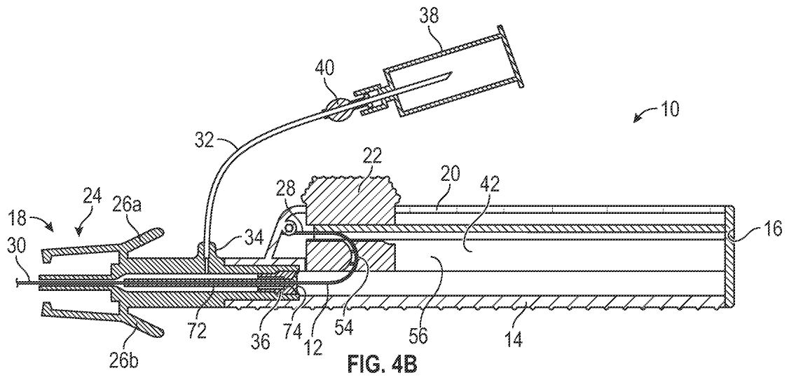

Referring now to FIGS. 4A-4B, in some embodiments, the septum 36 may be secured within the housing 14, and a proximal end of the septum 36 may include a tapered pocket 68, which may include an annular taper. In some embodiments, the instrument advancement device 10 may include a funnel-shaped element 70, which may include a distal column 72 coupled to a proximal cone-shape 74 or flared end. In some embodiments, the instrument 12 may extend through the funnel-shaped element 70. In some embodiments, when the advancement element 22 is moved linearly along the slot 20 from the retracted position to the advanced position, the distal column 72 may be configured to extend through the septum 36 and the proximal cone-shape 74 may be disposed within the tapered pocket 68 of the septum 36. In some embodiments, the distal column 72 or tube may prevent buckling of the instrument 12. In some embodiments, the proximal cone-shape 74 may facilitate securement of the funnel-shaped element 70 within the septum 36 when the advancement element 22 is in the advanced position.

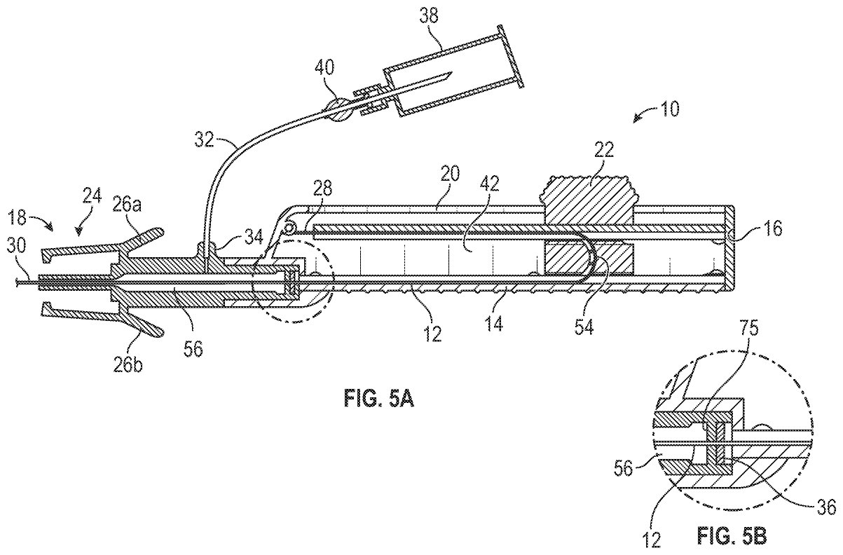

Referring now to FIGS. 5A-5B, in some embodiments, the housing 14 may include a support wall 75, which may include an opening 76 configured to receive the instrument 12, which may include a guidewire, therethrough. In some embodiments, the support wall 75 may be monolithically formed as a single unit with the connector 24 and/or an outer wall of the housing 14, which may provide stability to the support wall 75. In some embodiments, the support wall 75 may be generally transverse to a longitudinal axis of the housing 14.

In some embodiments, the distal face of the septum 36 may be in contact with the support wall 75 and supported by the support wall 75. In some embodiments, the septum 36 may be configured to prevent fluid leakage through the opening 76. In some embodiments, the instrument 12 may include the first end 28 and the second end 30. In some embodiments, when the advancement element 22 is moved linearly along the slot 20 from the retracted position to the advanced position, the second end 30 of the instrument 12, such as the guidewire, may be advanced beyond the distal end 18 of the housing 14.

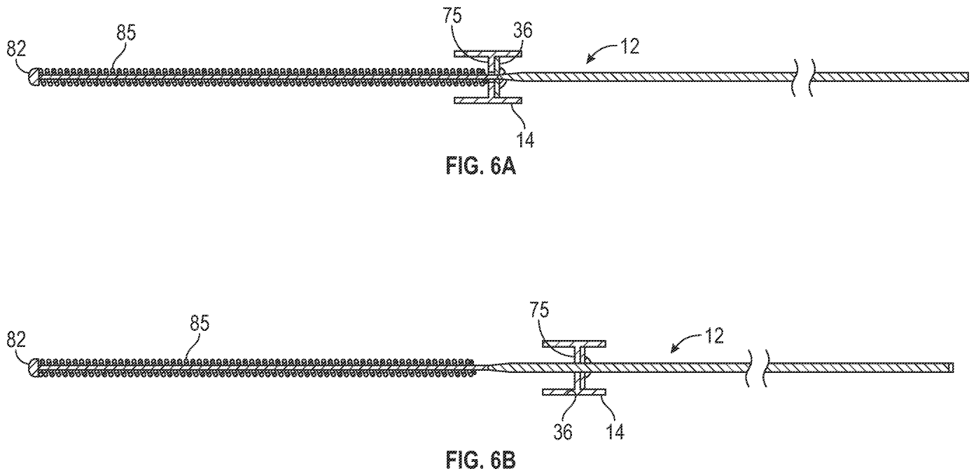

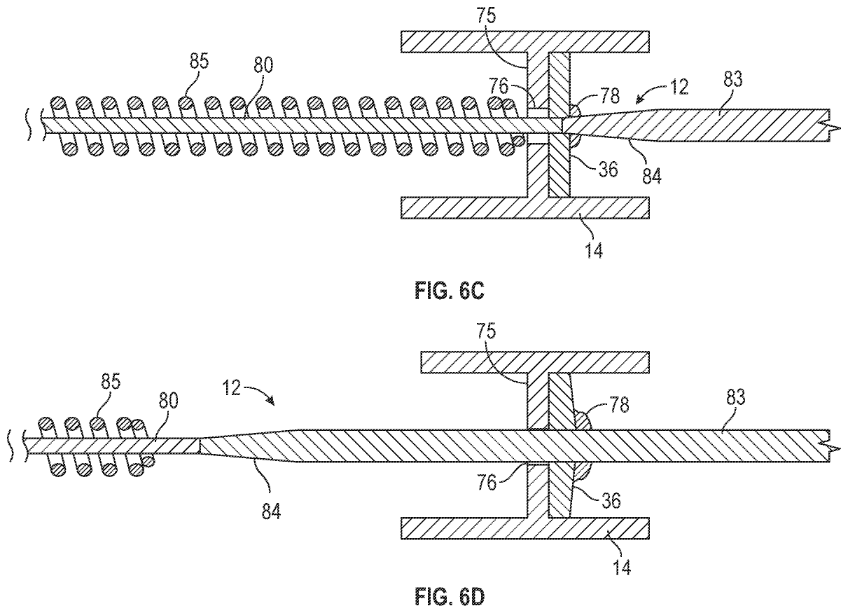

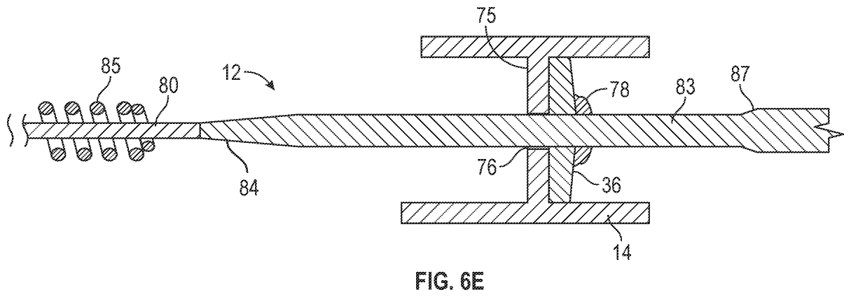

Referring now to FIGS. 6A-6E, in some embodiments, an outer diameter of the guidewire may increase in a proximal direction such that the guidewire compresses the septum 36 a greater amount when the advancement element 22 is in the retracted position than when the advancement element 22 is in the advanced position. In some embodiments, when the advancement element 22 is in the retracted position, the guidewire may extend through the septum 36 and the outer diameter of the guidewire may be spaced apart from the septum 36.

In some embodiments, a proximal face of the septum 36 may include a lubricant 78 extending over the opening 76. In some embodiments, the lubricant 78 may buildup or pool on the proximal face of the septum 36 in response to the instrument 12 being advanced distally, which may facilitate a seal around the instrument 12. In some embodiments, the lubricant 78 may include a silicon lubricant.

In some embodiments, the second end 30 may include a coil 85 extending around a portion 80 of the guidewire, which may have a uniform outer diameter. In some embodiments, the coil 85 may facilitate removal of blood clots or a thrombus. In some embodiments, the second end 30 may include a blunt tip 82, which may decrease a risk of damaging the vasculature of the patient.

In some embodiments, another portion 83 of the guidewire proximal to the portion 80 may include a taper 84, which may include the outer diameter of the guidewire increasing in the proximal direction such that the guidewire compresses the septum 36 a greater amount when the advancement element 22 is in the advanced position than when the advancement element 22 is in the retracted position. In some embodiments, the taper 84 may be gradual or steep.

In some embodiments, the other portion 83 of the guidewire proximal to the portion 80 may include multiple tapers. For example, the other portion 83 may include the taper 84 and/or a taper 87. In some embodiments, the taper 84 and/or the taper 87 may be monolithically formed as a single unit with a remaining portion of the guidewire. In some embodiments, the taper 84 and/or the taper 87 may be formed with a thin coating, a sleeve, or another additive process. In some embodiments, the guidewire may initially have clearance with the opening 76 when the advancement element 22 is in the retracted position.

In some embodiments, the guidewire may be configured to fully compress the septum 36 in response to the guidewire being significantly or completely advanced, when a risk of buckling due to a long unsupported shaft of the guidewire may be reduced. In some embodiments, maximum drag and maximum sealing of the septum 36 on the guidewire may not occur until the guidewire is significantly or completely advanced and the septum 36, when the septum 36 is fully compressed. In some embodiments, full compression of the septum 36 in response to the guidewire being significantly or completely advanced may occur as the result of the taper 87 or a single taper that is gradual and/or long.

In some embodiments, in response to the guidewire being advanced distally a first distance through the septum 36, the portion 80 may be disposed within the septum 36 and may provide slight compression of the septum 36 to create a weak fluid seal, sealing off a fluid pathway within a distal portion of the housing 14 and/or the extension tube 32 (see, for example, FIG. 2A). In some embodiments, in response to the guidewire being advanced distally a second distance through the septum 36 that is greater than the first distance, an increased outer diameter of the guidewire at or just proximal to the taper 84 may provide increased compression of the septum 36 and a stronger seal than the portion 80. In some embodiments, in response to the guidewire being advanced distally a third distance through the septum 36 that is greater than the second distance, an increased outer diameter at or just proximal to the taper 87 may provide an even tighter seal than the increased outer diameter of the guidewire at or just proximal to the taper 84 and/or may fully compress the septum 36. In some embodiments, the single taper that is gradual and/or long may provide the even tighter seal and/or may fully compress the septum 36.

In some embodiments, a seal between the guidewire and the opening 76 may increase in response to partial, near fully, or full advancement of the guidewire in the distal direction due to an increasing outer diameter of the guidewire. Although only a portion of the housing 14 is illustrated in FIGS. 6A-6D, the housing 14 may be similar to FIGS. 1-7.

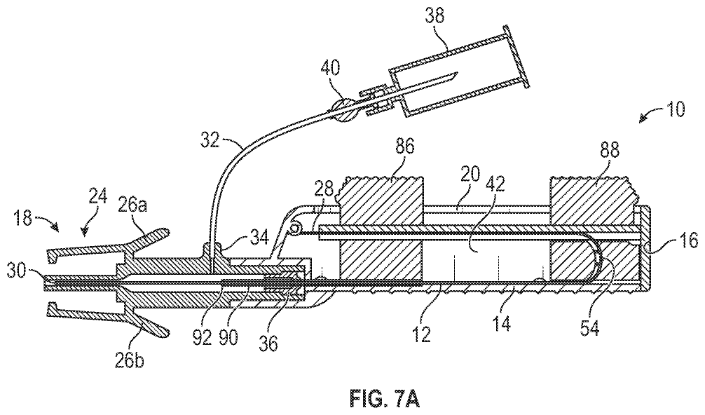

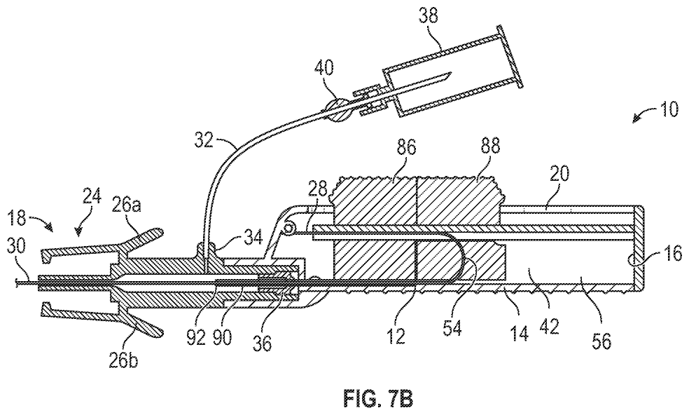

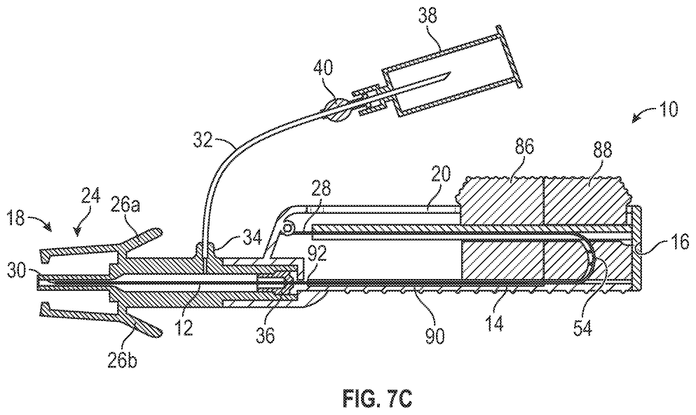

Referring now to FIGS. 7A-7C, in some embodiments, the instrument advancement device 10 may include a first advancement element 86 extending through the slot 20 and configured to move linearly along the slot 20 between a first retracted position and a first advanced position. In some embodiments, the instrument advancement device 10 may include a second advancement element 88 extending through the slot 20 and configured to move linearly along the slot 20 between a second retracted position and a second advanced position. In some embodiments, the second advancement element 88 may be proximal to the first advancement element 86.

In some embodiments, a tube 90 may be coupled to the first advancement element 86 and may be rigid or semi-rigid to facilitate movement through the septum 36. In some embodiments, when the first advancement element 86 is in the first advanced position, the tube 90 may extend through the septum 36, which may be secured within the housing 14. In some embodiments, the guidewire may be coupled to or extend through the second advancement element 88 and the tube 90. In some embodiments, in response to the first advancement element 86 being in the first retracted position, a distal end 92 of the tube 90 may be proximal to the septum 36. In some embodiments, the second advancement element 88 may be disposed in the retracted position. In some embodiments, the instrument advancement device 10 may be moved from the position in FIG. 7A to the position in FIG. 7B and then to the position in FIG. 7C.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed is:

1. An instrument advancement device, comprising:
a housing, comprising a proximal end, a distal end, a lumen disposed between the proximal end and a distal end, a slot disposed between the proximal end and the distal end, and a septum pocket disposed within the housing and configured to receive a septum;
an advancement element extending through the slot and configured to move linearly along the slot between a retracted position and an advanced position;
an instrument comprising a first end and a second end, wherein when the advancement element is moved linearly along the slot from the retracted position to the advanced position, the second end of the instrument is advanced beyond the distal end of the housing; and
the septum coupled to the instrument, wherein when the advancement element is moved linearly along the slot from the retracted position and the advanced position, the septum is configured to insert into the septum pocket to seal a portion of the lumen proximal to the septum.

2. The instrument advancement device of claim 1, wherein the distal end comprises a connector configured to couple to a catheter assembly.

3. The instrument advancement device of claim 1, wherein the instrument comprises a guidewire.

4. The instrument advancement device of claim 1, further comprising an extension tube extending from the distal end of the housing distal to the septum pocket.

5. The instrument advancement device of claim 1, wherein the septum comprises an annular taper, wherein the septum pocket comprises another annular taper.

6. The instrument advancement device of claim 1, wherein a cross-section of the septum is an oval or circular shape.

7. The instrument advancement device of claim 1, wherein a cross-section of the septum is a rectangular or square shape.

8. An instrument advancement device, comprising:
a housing, comprising a proximal end, a distal end, a lumen disposed between the proximal end and a distal end, a slot disposed between the proximal end and the distal end, and a septum pocket disposed within the housing and configured to receive a split septum;
an advancement element extending through the slot and configured to move linearly along the slot between a retracted position and an advanced position;
an instrument comprising a first end and a second end, wherein when the advancement element is moved linearly along the slot from the retracted position to the advanced position, the second end of the instrument is advanced beyond the distal end of the housing; and
the split septum proximal to the septum pocket and spaced apart from the septum pocket, wherein when the advancement element is moved linearly along the slot, the split septum is configured to insert into the septum pocket and the split septum is configured to compress to form a seal around the instrument and sealing a portion of the lumen proximal to the septum.

9. The instrument advancement device of claim 8, wherein the distal end comprises a connector configured to couple to a catheter assembly.

10. The instrument advancement device of claim 8, further comprising a spring disposed within the housing and proximate a proximal end of the split septum, wherein when the advancement element is moved linearly along the slot from the retracted position, the spring is configured to compress the split septum before the split septum is inserted into the septum pocket.

11. The instrument advancement device of claim 8, wherein the instrument comprises a guidewire.

12. An instrument advancement device, comprising:
a housing, comprising a proximal end, a distal end, a lumen disposed between the proximal end and a distal end, a slot disposed between the proximal end and the distal end;
an advancement element extending through the slot and configured to move linearly along the slot between a retracted position and an advanced position;
an instrument comprising a first end and a second end, wherein when the advancement element is moved linearly along the slot from the retracted position to the advanced position, the second end of the instrument is advanced beyond the distal end of the housing; and
a septum secured within the housing, wherein a proximal end of the septum comprises a tapered pocket;
a funnel-shaped element comprising a distal column coupled to a proximal cone-shape, wherein the instrument extends through the funnel-shaped element, wherein when the advancement element is moved linearly along the slot from the retracted position to the advanced position, the distal column is configured to extend through the septum and the proximal cone-shape is disposed within the tapered pocket of the septum.

13. The instrument advancement device of claim 12, wherein the distal end comprises a connector configured to couple to a catheter assembly.

14. The instrument advancement device of claim 12, wherein the instrument comprises a guidewire.

15. An instrument advancement device, comprising:
a housing, comprising a proximal end, a distal end, a slot disposed between the proximal end and the distal end, and a support wall, wherein the support wall comprises an opening configured to receive a guidewire therethrough;
a septum disposed within the housing, wherein a distal face of the septum is in contact with the support wall;
an advancement element extending through the slot and configured to move linearly along the slot between a retracted position and an advanced position; and
the guidewire comprising a first end and a second end, wherein when the advancement element is moved linearly along the slot from the retracted position to the advanced position, the second end of the guidewire is advanced beyond the distal end of the housing, wherein an outer diameter of the guidewire increases in a distal direction such that the guidewire compresses the septum a greater amount when the advancement element is in the retracted position than when the advancement element is in the advanced position.

16. The instrument advancement device of claim 15, wherein a proximal face of the septum comprises a lubricant.

17. The instrument advancement device of claim 16, wherein when the advancement element is in the retracted position, the guidewire extends through the septum and the outer diameter of the guidewire is spaced apart from the septum.

18. An instrument advancement device, comprising:
a housing, comprising a proximal end, a distal end, a lumen disposed between the proximal end and a distal end, a slot disposed between the proximal end and the distal end;
a septum disposed within the housing;
a first advancement element extending through the slot and configured to move linearly along the slot between a first retracted position and a first advanced position;
a second advancement element extending through the slot and configured to move linearly along the slot between a second retracted position and a second advanced position, wherein the second advancement element is proximal to the first advancement element;
a tube coupled to the first advancement element, wherein when the first advancement element is in the first advanced position, the tube extends through the septum; and
a guidewire coupled to or extending through the second advancement element and the tube, wherein the guidewire comprises a first end and a second end, wherein when the second advancement element is moved linearly along the slot from the retracted position to the advanced position, the second end of the guidewire is advanced beyond the distal end of the housing.

19. The instrument advancement device of claim 18, wherein when the first advancement element is in the first retracted position, a distal end of the tube is proximal to the septum.

20. The instrument advancement device of claim 19, wherein the second advancement element is disposed in the retracted position.
